(19) United States
(12) Patent Application Publication
(10) Pub. No.: US 2009/0192485 A1
(43) Pub. Date: Jul. 30, 2009

(54) SNARE DEVICE

(76) Inventor: Richard R. Heuser, Phoenix, AZ (US)

Correspondence Address: KOLISCH HARTWELL, P.C., 200 PACIFIC BUILDING, 520 SW YAMHILL STREET, PORTLAND, OR, 97204, US

(21) Appl. No.: 12/021,204

(22) Filed: Jan. 28, 2008

(51) Int. Cl. A61B 17/22; A61M 25/10

(52) U.S. Cl. 604/500; 604/96.01; 606/209

(57) ABSTRACT

An apparatus and method for removing an object from a tubular vessel are disclosed. The apparatus includes an expandable catheter and a plurality of wires configured to snare an object within a tubular vessel. A distal end of the expandable catheter may be manipulable between a first configuration wherein an opening thereon has a first diameter, and a second configuration wherein the opening has a second diameter which is larger than the first diameter. The apparatus may further include an inner catheter within its lumen, the inner catheter being extendable from the expandable catheter's lumen. The apparatus may additionally or alternatively include an outer catheter having a lumen from which the expandable catheter may be expanded.

### BACKGROUND

The removal of objects from tubular vessels within a living body can be difficult. Examples of naturally occurring objects which can be difficult to remove include thrombi or clots, gall stones, and urethral (kidney) stones. Examples of artificial objects which can be difficult to remove include valvular prostheses, closure devices for patent foramen ovale (PFO), atrial septal defect devices, ventricular septal defect devices, and the like. The latter can be especially difficult to remove when those objects expand during placement. When removing objects from tubular vessels in living bodies, thin-walled lumen (e.g., urethra and renal parenchyma) and thin-walled arteries (e.g., pulmonary or cerebral arteries) can be traumatized.

### SUMMARY OF THE DISCLOSURE

An apparatus for removing an object from a tubular vessel is disclosed, the apparatus comprising: an expandable catheter having a first distal end insertable to a first position adjacent to the object within the tubular vessel, a first opening adjacent to the first distal end, and a first lumen in communication with the first opening, the first distal end being manipulable between a first configuration where the first opening has a first diameter and a second configuration where the first opening has a second diameter which is greater than the first diameter; and a plurality of wires having a plurality of distal ends, wherein the plurality of wires is manipulable between a second position wherein the plurality of distal ends are within the first lumen, and a third position wherein the plurality of distal ends are extended out of the first lumen through the first opening, the plurality of wires being configured to snare the object when the plurality of wires are in the third position.

Additionally, a method of removing an object from a tubular vessel is disclosed, the method comprising the steps of: providing an expandable catheter having a first distal end, a first opening adjacent to the first distal end, and a first lumen in communication with the first opening; providing a plurality of wires having a plurality of distal ends; inserting the expandable catheter to a first position wherein the first distal end is adjacent to the object within the tubular vessel; manipulating the first distal end from a first configuration where the first opening has a first diameter to a second configuration where the first opening has a second diameter which is greater than the first diameter; advancing the plurality of wires through the first lumen so that the plurality of distal ends extend out of the first lumen through the first opening; and snaring the object with the plurality of distal ends.

### BRIEF DESCRIPTION OF THE DRAWINGS

FIG. 1A depicts an embodiment being traversed through a tubular vessel towards an object.

FIG. 1B depicts the embodiment of FIG. 1A deployed to snare an object.

FIG. 2 depicts an alternative embodiment, partially deployed, comprising two catheters.

FIG. 3 depicts an alternative embodiment, fully deployed, comprising two catheters snaring an object.

FIGS. 4A-F depict stages of an embodiment being used to remove an object from a tubular vessel.

FIG. 5 is a cross-sectional view of the embodiment shown in FIG. 4A, as viewed from the plane in 4A labeled Y.

### DETAILED DESCRIPTION

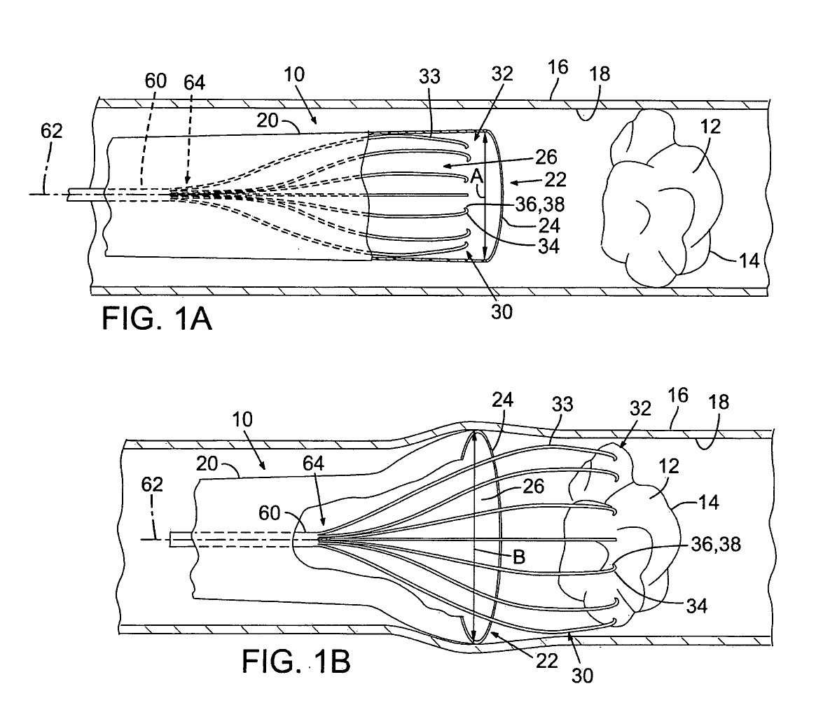

Referring to FIGS. 1A and B, an apparatus 10 for removing an object 12 having an outer surface 14 from a tubular vessel 16 is shown. Vessel 16 may have an inner wall 18. Apparatus 10 may comprise an expandable catheter 20, which may also be referred to as a middle catheter 20. Expandable catheter 20 may include a distal end 22, an opening 24 adjacent to distal end 22, and a lumen 26 traversing the length of expandable catheter 20 and terminating or being in communication with opening 24.

Distal end 22 of expandable catheter 20 may be insertable to a position adjacent to object 12 within tubular vessel 16. Distal end 22 of expandable catheter 20 may be manipulable between a first configuration where opening 24 has a first diameter labeled ‘A’ in FIG. 1A, and a second configuration where opening 24 has a second diameter labeled ‘B’ in FIG. 1B. The second diameter B may be greater than the first diameter, as seen in FIGS. 1A and B. Opening 24 may be expanded to the second diameter B in order to receive object 12, as will be discussed further below.

Apparatus 10 may include a plurality 30 of wires having a plurality 32 of distal ends. Various numbers of wires may be included. In some embodiments, the plurality 30 of wires may include as many as sixteen wires. Each wire may have a main portion 33 and an individual distal end 34.

Plurality 30 of wires may be manipulable between a various positions. For instance, plurality 30 of wires may be manipulated (i.e., advanced and retracted) between a position wherein the plurality 32 of distal ends are within lumen 26, as shown in FIG. 1A, and another position wherein the plurality 32 of distal ends are extended out of lumen 26 through opening 24, as shown in FIG. 1B. When extended out of lumen 26 through opening 24, the plurality 30 of wires may be configured to snare object 12 as shown in FIG. 1B. In some embodiments, plurality 32 of distal ends may be biased away from one another (e.g., as seen in FIG. 1B).

In some embodiments, each distal end 34 may terminate in an angled portion 36 which may form an angle with a main portion 33, as shown in FIGS. 1A-B, 2, 3, 4B-F and 5. These angles may be any angle between 0° and 180°, such as 30°, 45°, 90°, or 120°. The angled portions 36 may be configured to penetrate the surface 14 of object 12, snaring object 12 similarly to a hook. Each angled portion 36 further may be positioned so that it will not contact inner wall 18 of tubular vessel 16, thereby preventing trauma to vessel 16.

Additionally, in some embodiments, distal end 22 of expandable catheter 20 may be configured to compress object 12 when manipulated from the second configuration to the first configuration.

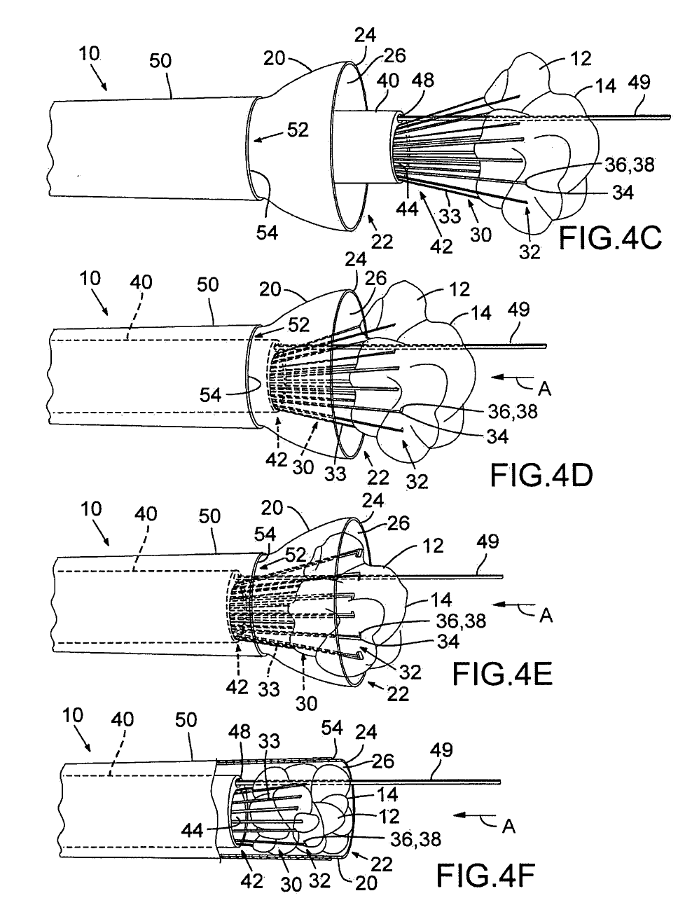

In some embodiments, plurality 30 of wires may be configured to, after snaring object 12, move object 12 through opening 24 at least partially into lumen 26. This may occur when the plurality 30 of wires is manipulated from the position where the plurality 32 of distal ends are extended out of lumen 26 back into the position where the plurality 32 of distal ends are within lumen 26, similar to that shown in FIGS. 4C-F.

In some embodiments, each distal end 34 may include a magnetic portion 38. In situations where object 12 includes ferromagnetic materials, as might be the case where object 12 comprises medical equipment, the magnetic portions 38 may attract object 12 towards them, assisting in the snaring of object 12. Although shown as being in the same area of each wire as angled portion 36, magnetic portion 38 may be anywhere on the wire, such as anywhere near distal end 34.

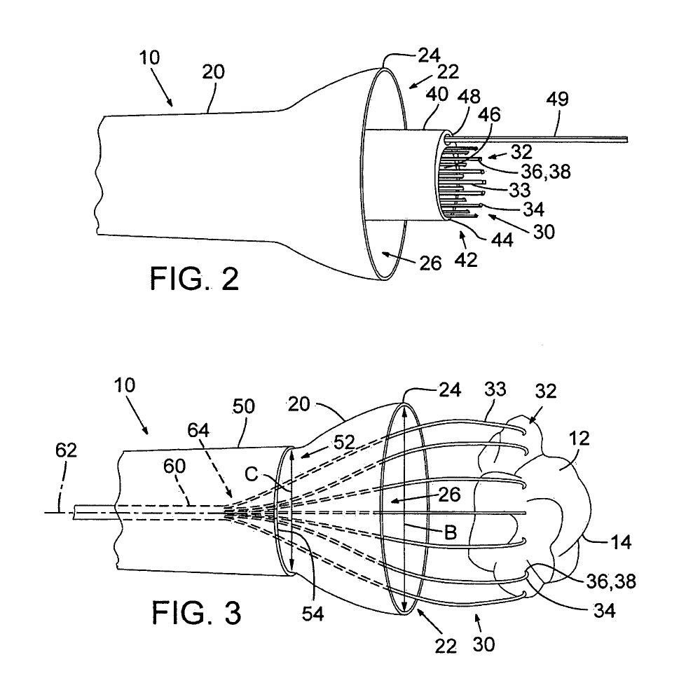

Referring now to FIGS. 2, 4A-F and 5, some embodiments of apparatus 10 may include an inner catheter 40. Inner catheter 40 may include a distal end 42, an opening 44 and a lumen 46 terminating with and being in communication with opening 44. Inner catheter 40 may be contained generally within lumen 26 of first catheter 20, as will be discussed further below. Some embodiments may further include a guidewire lumen 48, parallel to lumen 46, configured to receive a guidewire 49. In these embodiments, apparatus 10 may be advanced through vessel 16 over guidewire 49. Other embodiments not having an inner catheter 40 may also have a guidewire lumen 48.

Inner catheter 40 may be manipulable between a position where distal end 42 is within lumen 26 of expandable catheter 20, as seen in FIGS. 4A and 4D-F, and another position where distal end 42 extends out of lumen 26 through opening 24, as seen in FIGS. 2, 4B and 4C. Plurality 30 of wires may be further contained within lumen 46. More particularly, plurality 30 of wires may be manipulated between a position wherein the plurality 32 of distal ends are within lumen 46, as shown in FIG. 4A, and another position wherein the plurality 32 of distal ends are extended from lumen 46 through opening 44, as shown in FIGS. 2 and 4B-F.

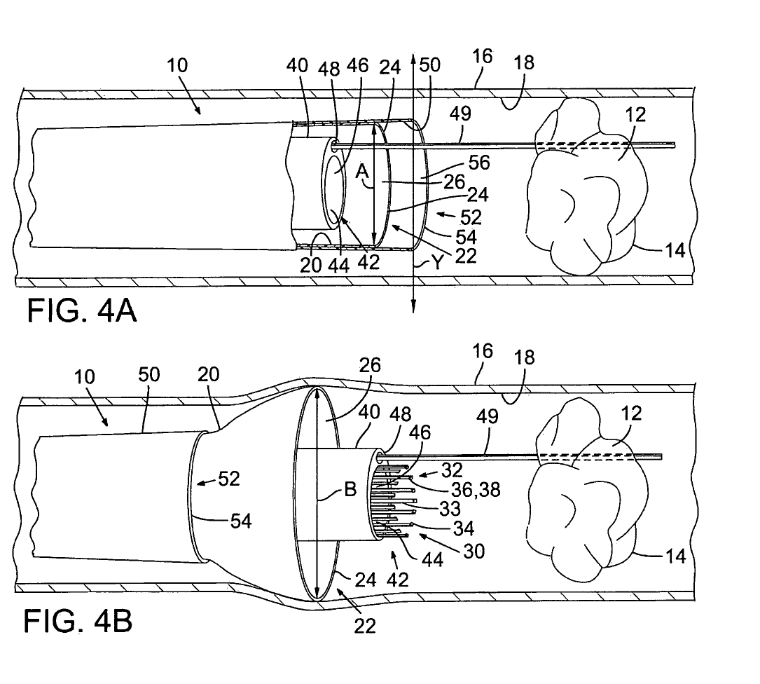

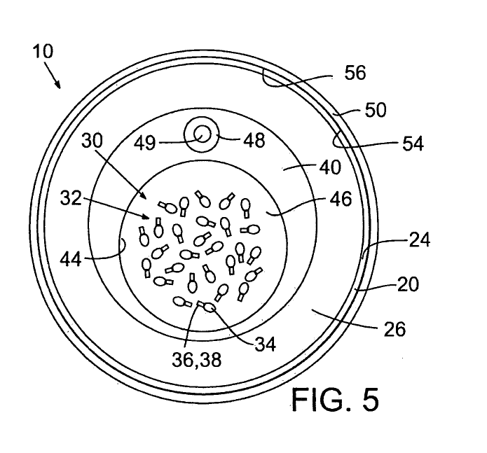

Referring now to FIGS. 3, 4A-F and 5, some embodiments of apparatus 10 may include an outer catheter 50 usable to manipulate distal end 22 of expandable catheter 20 between the first and second configurations. Outer catheter 50 may include a distal end 52, an opening 54 and a lumen 56 (seen in FIGS. 4A and 5) having a diameter (labeled ‘C’ in FIG. 3) which is less than the diameter of opening 24 of expandable catheter 20 when its distal end 22 is in the second configuration, as described above. Lumen 56 may terminate and be in communication with opening 54.

Expandable catheter 20 may be positioned within lumen 56 so that it is manipulable between various positions relative to outer catheter 50. For example, while apparatus 10 is being traversed through a vessel towards or away from object 12, expandable catheter 20 may be at a position where its distal end 22 is within lumen 56 of outer catheter 50 and is in the first configuration, as shown in FIGS. 4A and 5. Once apparatus 10 is in position adjacent to object 12, expandable catheter 20 may be manipulated to another position where its distal end 22 is extended out of lumen 56 through opening 54 and is in the second configuration, as shown in FIGS. 3 and 4B-E. In FIG. 4F, expandable catheter 20 is back in the first configuration, but not quite yet retracted fully into lumen 56 of outer catheter 50. While distal end 22 is shown in FIGS. 1B and 4B in the second configuration stretching the diameter of vessel 16, distal end 20 in the second configuration may alternatively have other diameters, such as a smaller diameter than vessel 16.

The manipulation of expandable catheter 20 between these two positions relative to outer catheter 50 may be accomplished by keeping outer catheter 50 stationary and extending expandable catheter 20 out of opening 54. Alternatively, expandable catheter 20 may be held stationary and outer catheter 50 may be withdrawn enough so that distal end 22 expands from the first configuration to the second configuration.

In some embodiments, such as those seen in FIGS. 1A-B and 3, apparatus 10 may include an inner wire 60 which forms an axis 62 extending through lumen 26 or lumen 46 (which is not shown). Plurality 30 of wires may include a plurality 64 of proximal ends each seamlessly bound to inner wire 60. In some embodiments, plurality 32 of distal ends may be nominally biased away from axis 62. In some embodiments, such as those shown in FIGS. 1A-B and 3, plurality 30 of wires may be curved. In other embodiments, such as those shown in FIGS. 2 and 4B-F, plurality 30 of wires may be straight.

Some embodiments of apparatus 10, such as those shown in FIGS. 4A-F and 5, may include all three of the expandable catheter 20, inner catheter 40 and outer catheter 50. In such embodiments, expandable catheter 20 may alternatively be referred to as middle catheter 20.

However, all three catheters are not required. As seen in FIGS. 1A and B, some embodiments comprise expandable catheter 20 with plurality 30 of wires extending from within lumen 26.

Other embodiments may include expandable catheter 20 and inner catheter 40 within lumen 26, as seen in FIG. 2. As described above, in such embodiments, plurality 30 of wires may be extendable directly from lumen 46 of inner catheter 40.

Yet other embodiments may comprise expandable catheter 20 and outer catheter 50, as seen in FIG. 3. In such embodiments, expandable catheter 20 may be extendable from within lumen 56, and plurality 30 or wires may be directly within lumen 26. Moreover, lumen 56 may have a diameter which is less than the diameter of opening 24 when distal end 22 of expandable catheter 20 is in the second configuration, as discussed above.

The disclosed apparatuses and methods are usable to remove objects from other tubular vessels within a living body such as blood vessels, urethras in the case of kidney stones, renal parenchyma, pulmonary/femoral/cerebral arteries or veins, biliary ducts in the case of gallstones, and other similar vessels.

It is believed that the following claims particularly point out certain combinations and subcombinations that are directed to one of the disclosed disclosures and are novel and non-obvious. Disclosures embodied in other combinations and subcombinations of features, functions, elements and/or properties may be claimed through amendment of the present claims or presentation of new claims in this or a related application. Such amended or new claims, whether they are directed to a different disclosure or directed to the same disclosure, whether different, broader, narrower or equal in scope to the original claims, are also included within the subject matter of the disclosures of the present disclosure. Where the claims recite “a” or “a first” element or the equivalent thereof, such claims include one or more such elements, neither requiring nor excluding two or more such elements. Further, ordinal indicators, such as first, second or third, for identified elements are used to distinguish between the elements, and do not indicate a required or limited number of such elements, and do not indicate a particular position or order of such elements unless otherwise specifically stated.

What is claimed is:

1. An apparatus for removing an object from a tubular vessel, the apparatus comprising:
an expandable catheter having a first distal end insertable to a first position adjacent to the object within the tubular vessel, a first opening adjacent to the first distal end, and a first lumen in communication with the first opening, the first distal end being manipulable between a first configuration where the first opening has a first diameter and a second configuration where the first opening has a second diameter which is greater than the first diameter; and
a plurality of wires having a plurality of distal ends, wherein the plurality of wires is manipulable between a second position wherein the plurality of distal ends are within the first lumen, and a third position wherein the plurality of distal ends are extended out of the first lumen through the first opening, the plurality of wires being configured to snare the object when the plurality of wires are in the third position.

2. The apparatus of claim 1 wherein the plurality of wires are further configured to move the object through the first opening at least partially into the first lumen when manipulated from the third position into the second position.

3. The apparatus of claim 2 wherein the first distal end is configured to compress the object when manipulated from the second configuration to the first configuration.

4. The apparatus of claim 1 further comprising an inner catheter having a second distal end, a second opening and a second lumen in communication with the second opening, the inner catheter being positioned within the first lumen so that it is manipulable between a fourth position where the second distal end is within the first lumen and a fifth position where the second distal end extends out of the first lumen through the first opening, wherein the plurality of distal ends are further contained within the second lumen when the plurality of wires is in the second position, and the plurality of distal ends are extended from the second lumen through the second opening when the plurality of wires is in the third position.

5. The apparatus of claim 4 wherein the inner catheter includes a guidewire lumen parallel to the second lumen.

6. The apparatus of claim 1 further comprising an outer catheter having a second distal end, a second opening, and a second lumen in communication with the second opening and having a third diameter smaller than the second diameter, wherein the expandable catheter is positioned within the second lumen so that it may be manipulated between a fourth position where the first distal end is within the second lumen and is in the first configuration, and a fifth position where the first distal end is extended out of the second lumen through the second opening and is in the second configuration.

7. The apparatus of claim 1 wherein the plurality of distal ends each terminates in an angled portion configured to penetrate the object's outer surface when the plurality of wires is in the third position.

8. The apparatus of claim 7 wherein each angled portion is positioned so that it will not contact an inner wall of the tubular vessel.

9. The apparatus of claim 1 wherein the plurality of distal ends each include a magnetic portion configured to attract ferromagnetic materials.

10. The apparatus of claim 1 further comprising an inner wire forming an axis extending through the first lumen, wherein the plurality of wires further includes a plurality of proximal ends each seamlessly bonded to the inner wire, the plurality of distal ends being nominally biased away from the axis.

11. An apparatus for removing an object from a tubular vessel, the apparatus comprising:
an outer catheter having a first distal end insertable to a first position adjacent to the object within the tubular vessel, a first opening, and a first lumen in communication with the first opening and having a first diameter;
a middle catheter having a second distal end, a second opening adjacent to the second distal end, and a second lumen in communication with the second opening, the second distal end being manipulable between a first configuration where the second opening has a second diameter and a second configuration where the second opening has a third diameter greater than the first diameter, the middle catheter being positioned within the first lumen so that it may be manipulated between a second position where the second distal end is within the first lumen and is in the first configuration, and a third position where the second distal end is extended out of the first lumen through the first opening and is in the second configuration;
an inner catheter having a third distal end, a third opening and a third lumen in communication with the third opening, the inner catheter being positioned within the second lumen so that it is manipulable between a fourth position where the third distal end is within the second lumen and a fifth position where the third distal end extends out of the second lumen through the second opening; and
a plurality of wires having a plurality of distal ends, wherein the plurality of wires is manipulable between a sixth position wherein the plurality of distal ends are within the third lumen, and a seventh position wherein the plurality of distal ends are extended out of the third lumen through the third opening, the plurality of wires being configured to snare the object when the plurality of wires are in the seventh position.

12. A method of removing an object from a tubular vessel, the method comprising the steps of:
providing an expandable catheter having a first distal end, a first opening adjacent to the first distal end, and a first lumen in communication with the first opening;
providing a plurality of wires having a plurality of distal ends;
inserting the expandable catheter to a first position wherein the first distal end is adjacent to the object within the tubular vessel;
manipulating the first distal end from a first configuration where the first opening has a first diameter to a second configuration where the first opening has a second diameter which is greater than the first diameter;
advancing the plurality of wires through the first lumen so that the plurality of distal ends extend out of the first lumen through the first opening; and
snaring the object with the plurality of distal ends.

13. The method of claim 12 further comprising the step of pulling the object through the first opening at least partially into the first lumen while the first distal in is in the second configuration.

14. The method of claim 13 further comprising the step of manipulating the first distal end from the second configuration to the first configuration to compress the object.

15. The method of claim 12 further comprising the steps of:
providing an inner catheter having a second distal end, a second opening and a second lumen in communication with the second opening; and
extending the inner catheter from a second position where the second distal end is within the first lumen to a third position where the second distal end extends out of the first lumen through the first opening;
wherein advancing the plurality of wires further includes advancing the plurality of wires through the second lumen so that the plurality of distal ends extend out of the second opening when the inner catheter is in the fifth position.

16. The method of claim 15 wherein extending the inner catheter includes a extending the inner catheter along a guidewire via a guidewire lumen disposed in the inner catheter parallel to the second lumen.

17. The method of claim 12 further comprising the steps of:
providing an outer catheter having a second distal end, a second opening, and a second lumen in communication with the second opening and having a third diameter smaller than the second diameter,
extending the expandable catheter from a second position where the first distal end is within the second lumen and is in the first configuration to a third position where the first distal end is extended out of the second lumen through the second opening and is in the second configuration.

18. The method of claim 12 wherein snaring the object further comprises penetrating the object's outer surface with an angled portion terminating each distal end of the plurality of distal ends.

19. The method of claim 18 wherein each angled portion is positioned so that it will not contact an inner wall of the tubular vessel.

20. The method of claim 12 wherein the object contains ferromagnetic material, the plurality of distal ends each include a magnetic portion configured to attract ferromagnetic materials, and snaring the object includes attracting the ferromagnetic material with the magnetic portions.
